(19) United States
(12) Patent Application Publication
(10) Pub. No.: US 2019/0276904 A1
(43) Pub. Date: Sep. 12, 2019

(54) METHODS AND DEVICES FOR DETECTING HEPATITIS C VIRUS

(71) Applicant: Quest Diagnostics Investments LLC, Secaucus, NJ (US)

(72) Inventors: Amy Rogers, San Juan Capistrano, CA (US); John Leake, Encinitas, CA (US); Nigel J. Clarke, Vista, CA (US); Russell E. Baumann, Aliso Viejo, CA (US)

(73) Assignee: Quest Diagnostics Investments LLC, Secaucus, NJ (US)

(21) Appl. No.: 16/349,976

(22) PCT Filed: Nov. 13, 2017
(86) PCT No.: PCT/US2017/061303

§ 371 Date: May 14, 2019

Related U.S. Application Data
(60) Provisional application No. 62/422,321, filed on Nov. 15, 2016.
(60) Provisional application No. 62/425,263, filed on Nov. 22, 2016.

(51) Int. Cl. C12Q 1/70; G01N 33/576

(52) U.S. Cl. CPC C12Q 1/707; C12Q 2600/158; G01N 33/5767

(57) ABSTRACT

The present disclosure provides rapid and non-invasive methods for determining whether a patient will benefit from treatment with therapeutic agents that inhibit Hepatitis C virus (HCV). These methods are based on detecting HCV RNA and/or anti-HCV antibodies in small-volume dried biological fluid samples that are collected using a microsampling device. Kits for use in practicing the methods are also provided.

### TECHNICAL FIELD

The present disclosure provides methods for determining whether a patient will benefit from treatment with therapeutic agents that inhibit Hepatitis C virus (HCV). These methods are based on detecting HCV RNA and/or anti-HCV antibodies in small-volume dried biological fluid samples that are collected using a microsampling device. Kits for use in practicing the methods are also provided.

### BACKGROUND

The following description of the background of the present disclosure is provided simply to aid the reader in understanding the disclosure and is not admitted to describe or constitute prior art to the present disclosure.

Hepatitis C virus (HCV) is considered to be the principal etiologic agent responsible for 90% to 95% of the cases of post-transfusion hepatitis (Rustgi V K. J Gastroenterol. 42:513-521 (2007), and the causative agent for most, if not all, blood-borne non-A, non-B hepatitis (NANBH). The presence of anti-HCV antibody indicates that an individual may have been infected with HCV and may be capable of transmitting HCV infection.

HCV is a single-stranded, positive sense RNA virus with a genome of approximately 9,500 nucleotides coding for 3,000 amino acids. As a blood-borne virus, HCV is transmitted by blood and blood products. The incidence of HCV infection is highest in association with intravenous drug abuse and to a lesser extent with other percutaneous exposures (Lauer G M & Walker B D, N Engl J Med 345:41-52 (2001)). According to the World Health Organization, about 130-170 million people are chronically infected with HCV, with more than 350,000 people dying from Hepatitis C-related liver diseases each year. The Centers for Disease Control estimates that 1.8% or 3.9 million Americans have been infected with Hepatitis C—of which 2.7 million are chronically infected. If left untreated, Hepatitis C can lead to liver cancer, liver damage and ultimately liver failure.

Thus, there is a substantial need for more robust and sensitive methods that can rapidly detect HCV in patients at risk for hepatitis C infection.

### SUMMARY

The present disclosure provides methods for detecting infection with HCV in patients with signs or symptoms of hepatitis, and in patients at risk for hepatitis C infection. The methods of the present technology may also be used to screen for hepatitis C infection in pregnant women to identify neonates who are at high risk of acquiring HCV during the prenatal period.

In one aspect, the present disclosure provides a method for detecting Hepatitis C virus (HCV) in a dried biological fluid sample comprising (a) extracting ribonucleic acids from a dried biological fluid sample eluted from an absorbent tip of a microsampling device; (b) reverse transcribing the extracted ribonucleic acids to generate a plurality of cDNA:RNA hybridization complexes; (c) amplifying the cDNA:RNA hybridization complexes with a primer pair that specifically hybridizes to the 5′ UTR of the HCV genome to produce HCV amplicons; and (d) detecting HCV in the dried biological fluid sample when the HCV amplicons produced in step (c) are detected. In some embodiments, the dried biological fluid sample is dried plasma, dried serum, or dried whole blood. The genotype of HCV present in the dried biological fluid sample may be one or more genotypes selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a. In certain embodiments, the dried biological fluid sample is isolated from a patient exhibiting signs or symptoms of hepatitis, or a patient at risk for HCV infection. In some embodiments, the cDNA:RNA hybridization complexes are amplified with Z05 or Z05D DNA polymerases.

Additionally or alternatively, in some embodiments, the dried biological fluid sample on the absorbent tip of the microsampling device is collected from a patient via fingerstick. In certain embodiments, the microsampling device is a MITRA® tip. Elution of the dried biological fluid sample may be performed by contacting the absorbent tip of the microsampling device with a lysis buffer. In some embodiments, the lysis buffer comprises guanidine isothiocyanate, and optionally β-mercaptoethanol. Additionally or alternatively, in some embodiments, elution of the dried biological fluid sample is performed by contacting the absorbent tip of the microsampling device with the lysis buffer for at least 30 minutes at 37° C. In some embodiments, the sample volume of the microsampling device is no more than 30 no more than 25 no more than 20 no more than 15 or no more than 10 μL.

In certain embodiments, the method further comprises contacting the cDNA:RNA hybridization complexes with a detectably labelled probe. In some embodiments, the detectable label is a fluorescent reporter selected from the group consisting of 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, BODIPY® R-6G, BOPIPY® 530/550, BODIPY® FL, Brilliant Yellow, Cal Fluor Red 610® (CFR610), coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′, 5″-dibromopyrogallol-sulfonephthalein (Bromopyrogallol Red), 7-di ethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, lanthamide phosphors, Malachite Green isothiocyanate, 4-methylumbelliferone, ortho cresolphthalein, nitrotyrosine, pararosaniline, Phenol Red, B-phycoerythrin, R-phycoerythrin, allophycocyanin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Quasar 670®, and VIC®.

In any of the above embodiments, the viral load of HCV in the dried biological fluid sample is at least 1-5 IU/mL, at least 5-10 IU/mL, at least 10-15 IU/mL, at least 15-20 IU/mL, at least 20-40 IU/mL, at least 40-60 IU/mL, at least 60-80 IU/mL, at least 80-100 IU/mL, at least 100-150 IU/mL, at least 150-200 IU/mL, at least 200-250 IU/mL, at least 250-300 IU/mL, at least 300-350 IU/mL, at least 350-400 IU/mL, at least 400-500 IU/mL, at least 500-600 IU/mL, at least 600-700 IU/mL, at least 700-800 IU/mL, at least 800-850 IU/mL, at least 850 IU/mL, or at least 900 IU/mL.

In another aspect, the present disclosure provides a method for detecting Hepatitis C virus (HCV) in a dried biological fluid sample comprising (a) eluting a dried biological fluid sample from an absorbent tip of a microsampling device; and (b) detecting HCV in the dried biological fluid sample when an anti-HCV antibody to at least one HCV encoded antigen is detected in the eluted dried biological fluid sample. In some embodiments, the dried biological fluid sample is dried plasma, dried serum, or dried whole blood. The genotype of HCV present in the dried biological fluid sample may be one or more genotypes selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a. In certain embodiments, the dried biological fluid sample is isolated from a patient exhibiting signs or symptoms of hepatitis, or a patient at risk for HCV infection. In some embodiments, the at least one HCV encoded antigen is selected from the group consisting of c22-3, c200, and NS5.

Additionally or alternatively, in some embodiments, the dried biological fluid sample on the absorbent tip of the microsampling device is collected from a patient via fingerstick. In certain embodiments, the microsampling device is a MITRA® tip. Elution of the dried biological fluid sample may be performed by contacting the absorbent tip of the microsampling device with a mixture comprising Phosphate Buffer Saline and 0.5% BSA. In certain embodiments, elution of the dried biological fluid sample is performed by contacting the absorbent tip of the microsampling device with the mixture comprising Phosphate Buffer Saline and 0.5% BSA for at least 2 hours at room temperature, or overnight at 2-8° C. In some embodiments, the sample volume of the microsampling device is no more than 30 no more than 25 no more than 20 no more than 15 or no more than 10 μL.

In one aspect, the present disclosure provides a method for selecting a patient exhibiting hepatitis symptoms for treatment with a therapeutic agent that inhibits HCV infection comprising (a) eluting a dried blood sample under conditions that result in the release of ribonucleic acids from blood cells, wherein the dried blood sample is collected from the patient with a microsampling device; (b) isolating ribonucleic acids from the eluted dried blood sample; (c) reverse transcribing the isolated ribonucleic acids to generate a plurality of cDNA:RNA hybridization complexes; (d) amplifying the cDNA:RNA hybridization complexes with a primer pair that specifically hybridizes to the 5′ UTR of the HCV genome to produce fluorescently labelled HCV amplicons; (e) detecting the fluorescently labelled HCV amplicons produced in step (d); and (f) selecting the patient for treatment with a therapeutic agent that inhibits HCV infection, if the fluorescently labelled HCV amplicons are detected.

Additionally or alternatively, in another aspect, the present disclosure provides a method for selecting a patient exhibiting hepatitis symptoms for treatment with a therapeutic agent that inhibits HCV infection comprising (a) eluting a dried blood sample with a buffer solution comprising 0.5% BSA, wherein the dried blood sample is collected from the patient with a microsampling device; (b) detecting an anti-HCV antibody to at least one HCV encoded antigen in the eluted dried blood sample; and (c) selecting the patient for treatment with a therapeutic agent that inhibits HCV infection, if the anti-HCV antibody is detected. In some embodiments, the at least one HCV encoded antigen is selected from the group consisting of c22-3, c200, and NS5. In some embodiments, the buffer solution further comprises Phosphate Buffer Saline.

In any of the above embodiments, the therapeutic agent that inhibits HCV infection is one or more agents selected from the group consisting of interferon alfacon-1, pegylated and/or non-pegylated interferon alfa-2b, peginterferon alfa-2a, ribavirin, telaprevir, boceprevir, sofosbuvir, simeprevir, daclatasvir, velpatasvir, ombitasvir, paritaprevir, ritonavir, dasabuvir, ledipasvir, elbasvir, danoprevir, grazoprevir, GS-7977, β-interferon, γ-interferon, amantadine, and 3TC. In some embodiments, the microsampling device is a MITRA® tip. The genotype of HCV present in the dried blood sample may be one or more genotypes selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a.

Also disclosed herein are kits for detecting the presence of Hepatitis C virus (HCV) in a dried biological fluid sample. In some embodiments, the kits comprise a microsampling device, a lysis buffer, reverse transcriptase, and optionally a primer pair that specifically hybridizes to the 5′ UTR of the HCV genome. In certain embodiments, the kits comprise a detectably labelled probe that specifically hybridizes to the 5′ UTR of the HCV genome.

Additionally or alternatively, in some embodiments, the kits comprise a microsampling device, PBS solution comprising 0.5% BSA, and a detectably labelled secondary antibody that specifically binds to an anti-HCV primary antibody. In certain embodiments, the kits further comprise a solid substrate comprising wells coated with at least one HCV encoded antigen selected from the group consisting of c22-3, c200, and NS5.

In any of the above embodiments of the kits of the present technology, the microsampling device is a MITRA® tip.

In one aspect, the present disclosure provides a method for detecting Hepatitis C virus (HCV) in a dried biological fluid sample comprising isolating HCV RNA from a dried biological fluid sample eluted from an absorbent tip of a microsampling device (e.g., MITRA® Tip) with a lysis buffer. In some embodiments, the HCV RNA is detected using reverse-transcription and real-time PCR. In certain embodiments, the lysis buffer comprises guanidine isothiocyanate, and optionally β-mercaptoethanol.

In another aspect, the present disclosure provides a method for detecting Hepatitis C virus (HCV) in a dried biological fluid sample comprising isolating an anti-HCV antibody from a dried biological fluid sample eluted from an absorbent tip of a microsampling device (e.g., MITRA® Tip) with a buffer solution comprising 0.5% BSA. In some embodiments, the buffer solution further comprises Phosphate Buffer Saline. In certain embodiments, the anti-HCV antibody binds to a HCV antigen selected from the group consisting of c22-3, c200, and NS5. The anti-HCV antibody may be detected using enzyme-linked immunosorbent assay (ELISA).

### BRIEF DESCRIPTION OF THE DRAWINGS

FIG. 1 shows the concordance between HCV RNA detection in dried blood samples collected on a MITRA® tip collection device via fingerstick from 12 patients and HCV RNA detection in the whole blood samples from the same patients collected using conventional blood draws.

FIG. 2 shows a correlation of recovered viral loads between fingerstick recovered HCV and serum recovered HCV.

### DETAILED DESCRIPTION

The present disclosure provides methods for determining whether a patient will benefit from treatment with therapeutic agents that inhibit HCV. These methods are based on detecting HCV RNA and/or anti-HCV antibodies in small-volume dried biological fluid samples that are collected using a microsampling device. Kits for use in practicing the methods are also provided. The methods disclosed herein are capable of detecting low viral loads in small-volume dried biological fluid samples obtained with a MITRA® Tip collection device via fingerstick. Further, it was determined that HCV RNA in dried biological fluid samples eluted from MITRA® tips remained stable, even when elution was performed 1 month after collection via fingerstick. This result was unexpected because the methods of the present technology do not entail pre-treating the MITRA® tips with additives such as urea, salts, chelators, or RNase inhibitors so as to prevent RNA degradation.

### Definitions

Unless otherwise defined, all technical and scientific terms used herein have the same meaning as commonly understood by one of ordinary skill in the art to which the present technology belongs. As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “about” in reference to a number is generally taken to include numbers that fall within a range of 1%-10% in either direction (greater than or less than) of the number unless otherwise stated or otherwise evident from the context.

As used herein, the “administration” of a therapeutic agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the terms “amplify” or “amplification” with respect to nucleic acid sequences, refer to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA and cDNA) or RNA. While the exemplary methods described hereinafter relate to amplification using polymerase chain reaction (PCR), numerous other methods such as isothermal methods, rolling circle methods, etc., are well known to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, Calif. 1990, pp 13-20; Wharam, et al., Nucleic Acids Res. 29(11):E54-E54 (2001).

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, the term “detecting” refers to determining the presence of a target HCV nucleic acid, or an antibody that specifically binds to a target HCV antigen in the sample. Detection does not require the method to provide 100% sensitivity and/or 100% specificity.

As used herein, “early virologic response (EVR)” means that a two-log or greater decrease in HCV RNA levels or undetectable levels of HCV RNA are observed in a patient after 12 weeks of therapy.

As used herein, the term “effective amount” refers to a quantity sufficient to achieve a desired therapeutic and/or prophylactic effect, e.g., an amount which results in the prevention of, or a decrease in HCV infection, or one or more symptoms associated with HCV infection. In the context of therapeutic or prophylactic applications, the amount of a therapeutic agent administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. As used herein, a “therapeutically effective amount” of a therapeutic drug or agent is meant levels in which the physiological effects of HCV infection are, at a minimum, ameliorated. A therapeutically effective amount can be given in one or more administrations.

As used herein, the terms “extraction” or “isolation” refer to any action taken to separate nucleic acids or proteins from other cellular material present in the sample. The term extraction or isolation includes mechanical or chemical lysis, addition of detergent or protease, or precipitation and removal of other cellular material.

The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In a preferred embodiment, the individual, patient or subject is a human.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides that function as primers or probes are generally at least about 10-15 nucleotides in length or up to about 70, 100, 110, 150 or 200 nucleotides in length, and more preferably at least about 15 to 25 nucleotides in length. Oligonucleotides used as primers or probes for specifically amplifying or specifically detecting a particular target nucleic acid generally are capable of specifically hybridizing to the target nucleic acid.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes.

As used herein, “rapid viral response (RVR)” means that undetectable levels of HCV RNA are observed in a patient after 4 weeks of therapy.

As used herein, the term “sample” refers to clinical samples obtained from a patient or isolated microorganisms. In preferred embodiments, a sample is obtained from a biological source (i.e., a “biological sample”), such as tissue, bodily fluid, or microorganisms collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include whole blood via fingerstick.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%). Exemplary sensitivities include at least 50, 60, 70, 80, 90, 95, 98, and 99%.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 85-95% and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not too} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include at least 50, 60, 70, 80, 90, 95, 98, and 99%.

As used herein, “specifically binds” refers to a molecule (e.g., an anti-HCV antibody) which recognizes and binds another molecule (e.g., a target HCV antigen), but that does not substantially recognize and bind other molecules. The terms “specific binding,” “specifically binds to,” or is “specific for” a particular molecule (e.g., a target HCV antigen), as used herein, can be exhibited, for example, by a molecule having a K_{d} for the molecule to which it binds to of at least about 10⁻⁴M, 10⁻⁵M, 10⁻⁶M, 10⁻⁷M, 10⁻⁸M, 10⁻⁹M, 10⁻¹⁰ M, 10⁻¹¹ M, 10⁻¹²M, or greater.

The term “stringent hybridization conditions” as used herein refers to hybridization conditions at least as stringent as the following: hybridization in 50% formamide, 5×SSC, 50 mM NaH₂PO4, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, “sustained virologic response (SVR)” means that undetectable levels of HCV RNA are observed in a patient after 24 weeks after the end of a therapeutic regimen.

As used herein “TaqMan® PCR detection system” refers to a method for real-time PCR. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

The terms “target nucleic acid” or “target sequence” as used herein refer to a nucleic acid sequence of interest to be detected and/or quantified in the sample to be analyzed. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

### HCV

The HCV virion is an enveloped positive-strand RNA virus in the Flaviviridae family with a genomic sequence of about 9600 bases which encodes a polyprotein of about 3,010 amino acids. In infected cells, the polyprotein is cleaved at multiple sites by cellular and viral proteases to produce the structural and non-structural (NS) proteins. The protein products of HCV include the structural proteins C, E1, and E2, and the non-structural proteins NS2, NS3, NS4A and NS4B, and NS5A and NS5B.

The nonstructural (“NS”) proteins are believed to provide the catalytic machinery for viral replication. In the case of HCV, the generation of mature nonstructural proteins (NS2, NS3, NS4A, NS4B, NS5A, and NS5B) is effected by two viral proteases. The first protease cleaves at the NS2-NS3 junction; the second one is a serine protease contained within the N-terminal region of NS3 (henceforth referred to as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites.

The NS4A protein appears to serve multiple functions, acting as a cofactor for the NS3 protease and possibly assisting in the membrane localization of NS3 and other viral replicase components. The complex formation of the NS3 protein with NS4A seems necessary to the processing events, enhancing the proteolytic efficiency at all of the sites. The NS3 protein also exhibits nucleoside triphosphatase and RNA helicase activities. The c200 protein is encoded by the putative NS3 and NS4 regions of the HCV genome.

NS5B is a RNA-dependent RNA polymerase that is involved in the replication of HCV. HCV NS5B polymerase is required for the synthesis of a double-stranded RNA from a single-stranded viral RNA that serves as a template in the replication cycle of HCV. Therefore, NS5B polymerase is considered to be an essential component in the HCV replication complex. (K. Ishi, et al, Hepatology 29: 1227-1235 (1999); V. Lohmann, et al., Virology 249: 108-118 (1998)). The c22-3 protein is encoded by the putative core region of the HCV genome.

### Microsampling Devices Employed in the Methods of the Present Technology

Conventional dried blood spotting techniques are accompanied by a number of drawbacks, including imprecise sample volume and reliance on a constant sample viscosity (i.e., the expectation that the sample will spread uniformly on the sample card). A constant viscosity results in blood spot diameters remaining constant when equal volume samples are administered to the cards. However, viscosity varies significantly between blood samples because of differing hematocrit (HCT) or packed cell volume (PCV) levels in the blood. Samples with high hematocrit levels form smaller diameter spots on the bloodspot papers, leading to different concentrations of blood within the fixed diameter of the spots sampled. PCV levels are believed to show a variance of about 45% in spot diameters. As internal standards are sprayed onto the spotted blood, this can result in a 45% error in quantitation. The microsampling devices employed in the methods disclosed herein confer several advantages, including the collection of more precise blood volumes, lack of hematocrit bias, and the ability to be easily automated with standard liquid handlers for lab processing.

Additionally, conventional blood spot techniques require a comparatively large volume of blood relative to the disclosed microsampling devices. A dried blood spot would generally require 50-75 μl per spot, while a microsampling device can yield results from approximately 20 μl. It has been recognized in the art that dried blood spots often have performance variability issues for detecting viral load compared to other samples types, such as plasma (Pannus et al., Medicine, 95:48(e5475) (2016)), and the volume of a dried blood spot may need to be significantly higher for certain types of assessment (e.g., optical density) compared to other sample types, such as serum (Brandao et al., J. Clin. Virol., 57:98-102 (2013)). Indeed, found that using both dried blood spot and plasma spot screening for detecting viral load and treatment failure in HIV patients receiving antiretroviral therapy found that both yielded a high rate of false positives (Sawadogo et al., J. Clin. Microbiol., 52(11):3878-83 (2014)).

The microsampling device useful in the methods of the present technology comprises an absorbent tip having a distal end and a proximal end. The width of the distal end of the absorbent tip is narrow compared to the width of the proximal end. The proximal end is attached to a holder, whereas the distal end is configured to contact a fluid to be absorbed, such as blood. The microsampling device permits biological fluid samples, such as blood, to be easily dried, shipped, and then later analyzed. In certain embodiments, the biological fluid is blood from a fingerstick.

Wicking action draws the blood into the absorbent tip. An optional barrier between the absorbent tip and the holder prevents blood from passing or wicking to the holder. The absorbent tip is composed of a material that wicks up substantially the same volume of fluid even when excess fluid is available (volumetric absorptive microsampling or VAMS™). The volume of the absorbent tip affects the volume of fluid absorbed. The size and shape of the absorbent tip may be varied to adjust the volume of absorbed blood and the rate of absorption. Blood volumes of about 7-15 μL, about 20 μL and even up to about 30 μL, may be acceptable. The sampling time may be about 2 seconds, about 3 seconds, about 5 seconds, or up to about 10 seconds.

In some embodiments, the material used for the absorbent tip is hydrophilic (e.g., polyester). Alternatively, the material may initially be hydrophobic and is subsequently treated to make it hydrophilic. Hydrophobic matrices may be rendered hydrophilic by a variety of known methods, such as plasma treatment or surfactant treatment (e.g., Tween-40 or Tween-80) of the matrix. In some embodiments, plasma treatment is used to render a hydrophobic material such as polyolefin, e.g., polyethylene, hydrophilic. Alternatively, the grafting of hydrophilic polymers to the surface and the chemical functionalization of active groups on the surface with polar or hydrophilic molecules such as sugars can be used to achieve a hydrophilic surface for the absorbent tip. Covalent modification could also be used to add polar or hydrophilic functional groups to the surface of absorbent tip.

In some embodiments, the microsampling device comprises an absorbent tip made of a hydrophilic polymeric material of sufficient size to absorb a maximum of about 20 μL of blood in about 2-5 seconds, and having a length of less than about 5 mm (0.2 inches) and a cross-sectional area of less than about 20 mm² and a density of less than about 4 g/cc. In some embodiments, the absorbent tips are composed of polyethylene and configured to absorb about 1-20 microliters of blood, preferably within 1-7 seconds, and more preferably within about 1-5 seconds. The absorbent tip may contain one or more of dried blood, or an internal standard.

In certain embodiments, the absorbent tips have a volume of about 35 mm³, absorb about 13-14 microliters of blood in about 3 seconds, absorb 9-10 microliters of blood in about 2.5 seconds, and have a pore volume of about 38% In other embodiments, the absorbent tips have a volume of about 24 microliters, a density of about 0.6 g/cc, absorb about 10 microliters of blood in about 2.5 seconds, and have a pore volume of about 40%. In some embodiments, the microsampling device is a MITRA® tip, as described in US 2013/0116597, which is herein incorporated by reference in its entirety.

The absorbent tip may be shaped with an exterior resembling a truncated cone with a narrow and rounded distal end. In some embodiments, the holder has a cylindrical post that fits into a recess inside the center of the absorbent tip and extending along the longitudinal axis of the absorbent tip and holder. The conical shape of the absorbent tip helps wick the sample quickly and uniformly.

The holder may be adapted for use with a pipette. In some embodiments, a tubular, conical shaped holder is preferred, with the absorbent tip on the narrow end of the holder. The wider opposite end of the holder may be closed, or open and hollow, and may optionally be configured to attach to a pipette tip. The holder may have outwardly extending flanges that are arranged to abut mating structures in holders, drying racks or test equipment to help position the absorbent tip at desired locations in such holders, drying racks and test equipment.

In certain embodiments, the holder may include a pipette tip or a tapering, tubular structure configured to nest with a pipette tip. The absorbent tip may be composed of polyethylene, and both the absorbent tip and holder are made under aseptic conditions, or are terminally sterilized. The absorbent tip may contain dried anti-coagulant. In some embodiments, the holder has a plurality of ribs extending along a length of the holder. The ribs may have a height and length selected to keep the absorbent tip from contacting walls of a recess into which the holder and absorbent tip are placed for shipment, or for extraction of the dried blood in the absorbent tip.

After absorbing a small-volume sample, the absorbent tip is then dried. In some embodiments, the small-volume blood sample is dried for at least 10 minutes, at least 20 minutes, at least 30 minutes, at least 40 minutes, at least 50 minutes, at least 1 hour, at least 2 hours, at least 3 hours, at least 4 hours, at least 5 hours, at least 6 hours, at least 8 hours, at least 12 hours, at least 16 hours, at least 20 hours, at least 24 hours, at least 48 hours, at least 72 hours, or at least 96 hours, or at least 1 week, or at least 2 weeks, or at least 3 weeks, or at least 4 weeks, or at least 1 month at ambient or room temperature. In certain embodiments, the small-volume blood sample is dried for about 2-3 hours.

Drying can be done on a suitable rack or holder, or preferably the absorbent tip and holder can be transferred to a special drying container configured to facilitate drying while minimizing contact between the absorbent tip and the walls of the drying container or other potential contaminant surfaces. The drying container may have a desiccant to facilitate drying. The drying container may also provide a protective cover which may be sealed for transport to prevent contamination. In some embodiments, the cover has a surface onto which printed indicia may be written to identify the source of the dried blood sample and provide other relevant information. In some embodiments, the dimensions of the container, and the relative positions of the holders within the container, will conform to SBS Microwell plate specifications. The microsampling device and the drying container may be placed in a plastic bag along with a desiccant to assist with drying and can either be shipped in this fashion, or shipped after the desiccant is removed.

In some embodiments, the wider opposite end of the holder is hollow and the container has a first portion with a mounting projection portion sized to fit into and releasably engage the hollow end of the holder. Additionally or alternatively, the container has a second portion releasably fastened to the first portion and has a recess configured to enclose a portion of the holder for transportation of the holder. The container may comprise a plurality of openings allowing air to access the absorbent tip of the microsampling device. Moreover, the first portion may have a side with an access port therein of sufficient size and located so that indicia may be applied through the port and onto the holder when the holder is on the mounting projection.

Upon receipt at the testing location, the absorbent tip may be eluted in a predetermined volume of a suitable buffer (as described herein) either manually or via automated means to extract the nucleic acids or proteins of interest from dried blood. Physical agitation techniques such as sonication or vortexing of the fluid and/or the absorbent tip may accelerate the extraction process from the dried blood into a liquid sample matrix. Physical separation techniques such as centrifugation, evaporation/reconstitution, concentration, precipitation, liquid/liquid extraction, and solid phase extraction can be used to further simplify the sample matrix for further analysis.

Each container may enclose a plurality of holders, wherein each holder comprises an absorbent tip at its distal end and has a hollow proximal end. The container likewise has a plurality of elongated mounting projections each sized to fit into and releasably engage the hollow ends of the plurality of holders. The second portion of the container has recesses configured to separately enclose each of the plurality of holders in a separate enclosure within the container. In certain embodiments, each of the plurality of holders has a plurality of ribs extending along a length of the holder with the ribs configured to keep the absorbent tip from contacting walls of the container. As desired, a desiccant may be placed inside the container to help dry the blood in the absorbent tip or maintain dryness. Each holder may have visible indicia associating the holder with the container and with at least one other holder, such as serial numbers with various portions of the number indicating related holders/absorbent tips and the container in which the holders are shipped.

### Real-Time PCR

Amplification of target nucleic acids (e.g., HCV RNA) can be detected by any of a number of methods well-known in the art such as gel electrophoresis, column chromatography, hybridization with a probe, sequencing, melting curve analysis, or “real-time” detection.

For real-time detection, primers and/or probes may be detectably labeled to allow differences in fluorescence when the primers become incorporated or when the probes are hybridized, for example, and amplified in an instrument capable of monitoring the change in fluorescence during the reaction. Real-time detection methods for nucleic acid amplification are well known and include, for example, the TaqMan® system, Scorpion™ primer system and use of intercalating dyes for double-stranded nucleic acid.

In real-time quantitative PCR, the accumulation of amplification product is measured continuously in both standard dilutions of target DNA and samples containing unknown amounts of target DNA. A standard curve is constructed by correlating initial template concentration in the standard samples with the number of PCR™ cycles (Ct) necessary to produce a specific threshold concentration of product. In the test samples, target PCR™ product accumulation is measured after the same Ct, which allows interpolation of target DNA concentration from the standard curve.

In some embodiments, amplified nucleic acids are detected by hybridization with a specific probe. Probe oligonucleotides, complementary to a portion of the amplified target sequence may be used to detect amplified fragments. In some embodiments, hybridization may be detected in real time. In an alternate embodiment, hybridization is not detected in real time. Amplified nucleic acids for each of the target sequences may be detected simultaneously (i.e., in the same reaction vessel such as multiplex PCR) or individually (i.e., in separate reaction vessels). In certain embodiments, multiple target nucleic acids are detected simultaneously, using two or more distinguishably-labeled (e.g., via different detectable moieties such as color), gene-specific oligonucleotide probes, one which hybridizes to the first target sequence and the other which hybridizes to the second target sequence.

In some embodiments, the different primer pairs are labeled with different distinguishable detectable moieties. Thus, for example, HEX and FAM fluorescent dyes may be present on different primer pairs in the multiplex PCR and associated with the resulting amplicons. In other embodiments, the forward primer is labeled with one detectable moiety, while the reverse primer is labeled with a different detectable moiety, e.g. FAM dye for a forward primer and HEX dye for a reverse primer. Use of different detectable moieties is useful for discriminating between amplified products which are of the same length or are very similar in length.

For sequence-modified nucleic acids, the target may be independently selected from the top strand or the bottom strand. Thus, all targets to be detected may comprise top strand, bottom strand, or a combination of top strand and bottom strand targets.

One general method for real-time PCR uses fluorescent probes such as the TaqMan® probes, molecular beacons, and Scorpion primer-probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion™ and TaqMan® technologies are based on the principle of fluorescence quenching and involve a donor fluorophore and a quenching moiety.

Real-time PCR is performed using any suitable instrument capable of detecting the accumulation of the PCR amplification product. Most commonly, the instrument is capable of detecting fluorescence from one or more fluorescent labels. For example, real-time detection on the instrument (e.g., an ABI Real-Time PCR System 7500® sequence detector) monitors fluorescence and calculates the measure of reporter signal, or Rn value, during each PCR cycle. The threshold cycle, or Ct value, is the cycle at which fluorescence intersects the threshold value. The threshold value can be determined by the sequence detection system software or manually.

In some embodiments, the probes employed are detectably labeled and the detecting is accomplished by detecting the probe label for each amplification product. A quencher may further be associated with the detectable label which prevents detection of the label prior to amplification of the probe's target. TaqMan® probes are examples of such probes.

TaqMan® probes (Heid et al., Genome Res. 6: 986-994, 1996) use the fluorogenic 5′ exonuclease activity of Taq polymerase to measure the amount of target sequences in DNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase replicates a template on which a TaqMan® probe is bound, its 5′ exonuclease activity cleaves the probe. This terminates the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye. If the quencher is an acceptor fluorophore, then accumulation of PCR product can be detected by monitoring the decrease in fluorescence of the acceptor fluorophore.

In certain embodiments, real-time PCR is performed using a bifunctional primer-probe detection system (e.g., Scorpion™ primers). With Scorpion primers, sequence-specific priming and PCR product detection is achieved using a single molecule. The Scorpion primer maintains a stem-loop configuration in the unhybridized state. The fluorophore is attached to the 5′ end and is quenched by a moiety coupled to the 3′ end, although in certain embodiments, this arrangement may be switched. The 3′ portion of the stem and/or loop also contains sequence that is complementary to the extension product of the primer and is linked to the 5′ end of a specific primer via a non-amplifiable monomer. After extension of the primer moiety, the specific probe sequence is able to bind to its complement within the extended amplicon, thus opening up the hairpin loop. This prevents the fluorescence from being quenched and a signal is observed. A specific target is amplified by the reverse primer and the primer portion of the Scorpion™ primer, resulting in an extension product. A fluorescent signal is generated due to the separation of the fluorophore from the quencher resulting from the binding of the probe element of the Scorpion™ primer to the extension product.

In some embodiments, the probes employed in the disclosed methods comprise or consist of short fluorescently labeled DNA sequences designed to detect sections of DNA sequence with a genetic variation such as those disclosed in French et al., Mol Cell Probes, 5(6):363-74 (2001), incorporated by reference herein in its entirety. HyBeacons® are an example of this type of probe.

In some embodiments of the method, at least one primer of each primer pair or at least one probe in the amplification reaction comprises a detectable moiety. Alternatively, the detectable moiety may be on a probe that is attached to the primer, such as with a primer-probe. In some embodiments, the detectable moiety or label is a fluorophore. Suitable fluorescent moieties include, but are not limited to the following fluorophores: 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, BODIPY® R-6G, BOPIPY® 530/550, BODIPY® FL, Brilliant Yellow, Cal Fluor Red 610® (CFR610), coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′, 5″-dibromopyrogallol-sulfonephthalein (Bromopyrogallol Red), 7-di ethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilb ene-2,2′-di sulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, lanthamide phosphors, Malachite Green isothiocyanate, 4-methylumbelliferone, ortho cresolphthalein, nitrotyrosine, pararosaniline, Phenol Red, B-phycoerythrin, R-phycoerythrin, allophycocyanin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Quasar 670®, and VIC®.

Suitable quenchers are selected based on the fluorescence spectrum of the particular fluorophore. Useful quenchers include, for example, the Black Hole™ quenchers BHQ-1, BHQ 2, and BHQ-3 (Biosearch Technologies, Inc.), and the ATTO-series of quenchers (ATTO 540Q, ATTO 580Q, and ATTO 612Q; Atto-Tec GmbH).

### Alternate Methods of Detecting Target Nucleic Acids

Alternatively, detection of the target nucleic acids (e.g., HCV RNA) can occur by measuring the end-point of the reaction. In end-point detection, the amplicon(s) could be detected by first size-separating the amplicons, and then detecting the size-separated amplicons. The separation of amplicons of different sizes can be accomplished by gel electrophoresis, column chromatography, capillary electrophoresis, or other separation methods known in the art.

The detectable label can be incorporated into, associated with or conjugated to a nucleic acid. The detectable label can be attached by spacer arms of various lengths to reduce potential steric hindrance or impact on other useful or desired properties. See, e.g., Mansfield, 9 Mol. Cell. Probes 145-156 (1995). Detectable labels can be incorporated into nucleic acids by covalent or non-covalent means, e.g., by transcription, such as by random-primer labeling using Klenow polymerase, or nick translation, or amplification, or equivalent as is known in the art. For example, a nucleotide base is conjugated to a detectable moiety, such as a fluorescent dye, and then incorporated into nucleic acids during nucleic acid synthesis or amplification.

Examples of other useful labels that aid in the detection of target nucleic acids include radioisotopes (e.g., ³²P, ³⁵S, ³H, ¹⁴C, ¹²⁵I, ¹³¹I) electron-dense reagents (e.g., gold), enzymes (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

In other embodiments, fluorescent nucleotide analogs can be used to label nucleic acids, see, e.g., Jameson, Methods. Enzymol. 278: 363-390 (1997); Zhu, Nucl. Acids Res. 22: 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, PNAS. 80: 1194 (1983).

### HCV Detection Assays of the Present Technology

Provided herein are methods for detecting Hepatitis C virus (HCV) in a dried biological fluid sample comprising isolating HCV RNA from a dried biological fluid sample eluted from an absorbent tip of a microsampling device (e.g., MITRA® Tip) with a lysis buffer. In some embodiments, the HCV RNA is detected using reverse-transcription and real-time PCR. In certain embodiments, the lysis buffer comprises guanidine isothiocyanate, and optionally β-mercaptoethanol.

Also disclosed herein are methods for detecting Hepatitis C virus (HCV) in a dried biological fluid sample comprising isolating an anti-HCV antibody from a dried biological fluid sample eluted from an absorbent tip of a microsampling device (e.g., MITRA® Tip) with a buffer solution comprising 0.5% BSA. In some embodiments, the buffer solution further comprises Phosphate Buffer Saline. In certain embodiments, the anti-HCV antibody binds to a HCV antigen selected from the group consisting of c22-3, c200, and NS5. The anti-HCV antibody may be detected using enzyme-linked immunosorbent assay (ELISA).

In one aspect, the present technology provides a method for detecting Hepatitis C virus (HCV) in a dried biological fluid sample comprising (a) extracting ribonucleic acids from a dried biological fluid sample eluted from an absorbent tip of a microsampling device; (b) reverse transcribing the extracted ribonucleic acids to generate a plurality of cDNA:RNA hybridization complexes; (c) amplifying the cDNA:RNA hybridization complexes with a primer pair that specifically hybridizes to the 5′ UTR of the HCV genome to produce HCV amplicons; and (d) detecting HCV in the dried biological fluid sample when the HCV amplicons produced in step (c) are detected. In some embodiments, the dried biological fluid sample is dried plasma, dried serum, or dried whole blood. The genotype of HCV present in the dried biological fluid sample may be one or more genotypes selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a. In certain embodiments, the dried biological fluid sample is isolated from a patient exhibiting signs or symptoms of hepatitis, or a patient at risk for HCV infection. In some embodiments, the cDNA:RNA hybridization complexes are amplified with Z05 or Z05D DNA polymerases.

Additionally or alternatively, in some embodiments, the dried biological fluid sample on the absorbent tip of the microsampling device is collected from a patient via fingerstick. In certain embodiments, the microsampling device is a MITRA® tip. Elution of the dried biological fluid sample may be performed by contacting the absorbent tip of the microsampling device with a lysis buffer. In some embodiments, the lysis buffer comprises guanidine isothiocyanate, and optionally β-mercaptoethanol. Additionally or alternatively, in some embodiments, elution of the dried biological fluid sample is performed by contacting the absorbent tip of the microsampling device with the lysis buffer for at least 30 minutes at 37° C. In some embodiments, the sample volume of the microsampling device is no more than 30 μL, no more than 25 μL, no more than 20 μL, no more than 15 μL, or no more than 10 μL. Further, it was determined that HCV RNA in dried biological fluid samples eluted from MITRA® tips remained stable, even when elution was performed 1 month after collection via fingerstick. This result was unexpected because the methods of the present technology do not entail pre-treating the MITRA® tips with additives such as urea, salts, chelators, or RNase inhibitors so as to prevent RNA degradation.

In certain embodiments, the method further comprises contacting the cDNA:RNA hybridization complexes with a detectably labelled probe. In some embodiments, the detectable label is a fluorescent reporter selected from the group consisting of 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, BODIPY® R-6G, BOPIPY® 530/550, BODIPY® FL, Brilliant Yellow, Cal Fluor Red 610® (CFR610), coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′, 5″-dibromopyrogallol-sulfonephthalein (Bromopyrogallol Red), 7-di ethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, lanthamide phosphors, Malachite Green isothiocyanate, 4-methylumbelliferone, ortho cresolphthalein, nitrotyrosine, pararosaniline, Phenol Red, B-phycoerythrin, R-phycoerythrin, allophycocyanin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Quasar 670®, and VIC®.

In any of the above embodiments, the viral load of HCV in the dried biological fluid sample is at least 1-5 IU/mL, at least 5-10 IU/mL, at least 10-15 IU/mL, at least 15-20 IU/mL, at least 20-40 IU/mL, at least 40-60 IU/mL, at least 60-80 IU/mL, at least 80-100 IU/mL, at least 100-150 IU/mL, at least 150-200 IU/mL, at least 200-250 IU/mL, at least 250-300 IU/mL, at least 300-350 IU/mL, at least 350-400 IU/mL, at least 400-500 IU/mL, at least 500-600 IU/mL, at least 600-700 IU/mL, at least 700-800 IU/mL, at least 800-850 IU/mL, at least 850 IU/mL, or at least 900 IU/mL.

In another aspect, the present disclosure provides a method for detecting Hepatitis C virus (HCV) in a dried biological fluid sample comprising (a) eluting a dried biological fluid sample from an absorbent tip of a microsampling device; and (b) detecting HCV in the dried biological fluid sample when an anti-HCV antibody to at least one HCV encoded antigen is detected in the eluted dried biological fluid sample. In some embodiments, the dried biological fluid sample is plasma, serum, or whole blood. The genotype of HCV present in the dried biological fluid sample may be one or more genotypes selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a. In certain embodiments, the dried biological fluid sample is isolated from a patient exhibiting signs or symptoms of hepatitis, or a patient at risk for HCV infection. In some embodiments, the at least one HCV encoded antigen is selected from the group consisting of c22-3, c200, and NS5.

Additionally or alternatively, in some embodiments, the dried biological fluid sample on the absorbent tip of the microsampling device is collected from a patient via fingerstick. In certain embodiments, the microsampling device is a MITRA® tip. Elution of the dried biological fluid sample may be performed by contacting the absorbent tip of the microsampling device with a mixture comprising Phosphate Buffer Saline and 0.5% BSA. In certain embodiments, elution of the dried biological fluid sample is performed by contacting the absorbent tip of the microsampling device with the mixture comprising Phosphate Buffer Saline and 0.5% BSA for at least 2 hours at room temperature, or overnight at 2-8° C. In some embodiments, the sample volume of the microsampling device is no more than 30 μL, no more than 25 μL, no more than 20 μL, no more than 15 μL, or no more than 10 μL.

While it is to be understood that, for the purposes of the disclosed methods, the biological fluid sample may comprise plasma, serum, or whole blood, those of skill in the art will know that in some instances, one sample type may be preferred over another. For instance, grossly hemolyzed whole blood samples may cause interferences in immunoassays that could compromise the results of the assay (Schiettecatta et al., Interferences in Immunoassays in ADVANCES IN IMMUNOASSAY TECHNOLOGY, (Norman H. L. Chiu ed. 2012). Accordingly, when performing certain immunoassays, plasma or serum may be preferred over whole blood. However, as shown in the results in Table 3 below, whole blood samples are stable even after long-term storage at room temperature when the sample was collected using a microsampling device.

### Treatment for HCV Infection

Disclosed herein are methods for determining whether a patient will benefit from treatment with therapeutic agents that inhibit HCV.

Examples of therapeutic agents that inhibit HCV include interferon alfacon-1, pegylated and/or non-pegylated interferon alfa-2b, peginterferon alfa-2a, ribavirin, telaprevir, boceprevir, sofosbuvir, simeprevir, daclatasvir, velpatasvir, ombitasvir, paritaprevir, ritonavir, dasabuvir, ledipasvir, elbasvir, danoprevir, grazoprevir, GS-7977, β-interferon, γ-interferon, amantadine, 3TC (also known as the (−) enantiomer of the nucleoside analogue cytosine-1,3-oxathiolane), and inhibitors that target the HCV life cycle, including but not limited to, helicase, polymerase, metalloprotease or internal ribosome entry site (IRES).

Examples of inhibitors that target the HCV life cycle include heterocyclic-substituted carboxamides (described in U.S. Pat. No. 5,633,388) that interfere with the helicase activity of the NS3 protein; the phenanthrenequinone reported in Chu et al., Tet. Lett. 7229-7232 (1996), which inhibits HCV NS3 protease in vitro; morpholinylcarbonyl-benzoyl-peptide analogues (WO 1995/33764); NS5A/5B substrate-based peptide analogues (WO 1998/17679); thiazolidine derivatives (Brown-Driver et al., Antiviral Research 30(1), A23 (1996)) which inhibit HCV protease; and other peptide inhibitors of HCV NS3 protease (Steinkühler et al., Biochemistry 37:8899-8905 (1998); Ingallinella et al., Biochemistry 37:8906-8914 (1998)).

In one aspect, the present disclosure provides a method for selecting a patient exhibiting hepatitis symptoms for treatment with at least one therapeutic agent that inhibits HCV infection comprising (a) eluting a dried blood sample under conditions that result in the release of ribonucleic acids from blood cells, wherein the dried blood sample is collected from the patient with a microsampling device; (b) isolating ribonucleic acids from the eluted dried blood sample; (c) reverse transcribing the isolated ribonucleic acids to generate a plurality of cDNA:RNA hybridization complexes; (d) amplifying the cDNA:RNA hybridization complexes with a primer pair that specifically hybridizes to the 5′ UTR of the HCV genome to produce fluorescently labelled HCV amplicons; (e) detecting the fluorescently labelled HCV amplicons produced in step (d); and (f) selecting the patient for treatment with a therapeutic agent that inhibits HCV infection, if the fluorescently labelled HCV amplicons are detected.

Additionally or alternatively, in one aspect, the present disclosure provides a method for selecting a patient exhibiting hepatitis symptoms for treatment with a therapeutic agent that inhibits HCV infection comprising (a) eluting a dried blood sample with a buffer solution comprising 0.5% BSA, wherein the dried blood sample is collected from the patient with a microsampling device; (b) detecting an anti-HCV antibody to at least one HCV encoded antigen in the eluted dried blood sample; and (c) selecting the patient for treatment with a therapeutic agent that inhibits HCV infection, if the anti-HCV antibody is detected. In some embodiments, the at least one HCV encoded antigen is selected from the group consisting of c22-3, c200, and NS5. In some embodiments, the buffer solution further comprises Phosphate Buffer Saline.

In any of the above embodiments, the therapeutic agent that inhibits HCV infection is one or more agents selected from the group consisting of interferon alfacon-1, pegylated and/or non-pegylated interferon alfa-2b, peginterferon alfa-2a, ribavirin, telaprevir, boceprevir, sofosbuvir, simeprevir, daclatasvir, velpatasvir, ombitasvir, paritaprevir, ritonavir, dasabuvir, ledipasvir, elbasvir, danoprevir, grazoprevir, GS-7977, β-interferon, γ-interferon, amantadine, 3TC, and inhibitors that target the HCV life cycle. In any of the above embodiments, the microsampling device is a MITRA® tip.

In certain embodiments, the HCV in the dried blood sample has at least one genotype selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a.

Hepatitis symptoms include, but are not limited to, flu-like symptoms, joint and muscle aches, mild skin rash, loss of appetite, abdominal pain, dark urine, grey colored stool, jaundice, chronic liver disease, cryoglobulinemia, pain and tenderness in the area of the liver, fever, weight loss, depression, and fatigue. Patients at risk for contracting HCV include those exposed to infectious blood or blood products, blood transfusion recipients, organ transplant recipients prior to 1992, HIV patients, long-term hemodialysis patients, subjects who have a history of unprotected sexual activity, individuals with tattoos and body piercings, patients exhibiting signs or symptoms of liver disease, illicit drug users, and patients born to mothers infected with HCV.

Quantitation of HCV RNA for measuring baseline viral loads and for treatment monitoring has been well established in demonstrating the efficacy of antiviral response to some combination therapy regimens (McHutchison J G et al., N Engl J Med 339:1485-1492 (1998); Davis G L et al., N Engl J Med 339:1493-1499 (1998); Manns M P et al., Lancet 358:958-965 (2001); Fried M W et al., N Engl J Med 347:975-982 (2002); Hadziyannis S J et al., Ann Intern Med 140:346-355 (2004)). Current guidelines for the management and treatment of HCV recommend quantitative testing for HCV RNA before the start of antiviral therapy, during therapy (response guided therapy), and generally 12 to 24 weeks following the end of treatment. Failure to achieve early virologic response (EVR) has a high negative predictive value for achieving a sustained virologic response (SVR) and is considered when determining whether a particular therapy should be terminated for a patient. A rapid viral response (RVR) has a high positive predictive value for SVR.

The present disclosure provides methods for evaluating the efficacy of a therapeutic regimen in a patient exhibiting signs or symptoms of HCV, or in a patient at risk for HCV infection by recurrently monitoring the levels of HCV RNA or anti-HCV antibodies in dried biological fluid samples (e.g., blood) collected from the patient with a microsampling device (e.g., via fingerstick). In some embodiments, the microsampling device is a MITRA® tip.

In one aspect, the present disclosure provides methods for evaluating the efficacy of a HCV therapeutic regimen on HCV infection in a patient comprising (a) eluting a first dried blood sample under conditions that result in the release of ribonucleic acids from blood cells, wherein the first dried blood sample is collected from the patient with a microsampling device prior to administrating the HCV therapeutic regimen to the patient; (b) detecting HCV RNA in the eluted first dried blood sample via reverse-transcription and real-time PCR; (c) eluting a second dried blood sample under conditions that result in the release of ribonucleic acids from blood cells, wherein the second dried blood sample is collected from the patient with a microsampling device following administration of the HCV therapeutic regimen to the patient; and (d) detecting HCV RNA in the eluted second dried blood sample via reverse-transcription and real-time PCR, wherein the HCV therapeutic regimen is identified as having a therapeutic effect on HCV infection if the HCV RNA levels in the second dried blood sample following the administration of the HCV therapeutic regimen are reduced compared to the HCV RNA levels observed in step (b). In certain embodiments, the patient may exhibit an early virologic response (EVR), rapid viral response (RVR), and/or sustained virologic response (SVR).

Additionally or alternatively, the present disclosure provides methods for evaluating the efficacy of a HCV therapeutic regimen on HCV infection in a patient comprising (a) eluting a first dried blood sample with a buffer solution comprising 0.5% BSA, wherein the first dried blood sample is collected from the patient with a microsampling device prior to administrating the HCV therapeutic regimen to the patient; (b) detecting at least one anti-HCV antibody to at least one HCV encoded antigen in the eluted first dried blood sample; (c) eluting a second dried blood sample with a buffer solution comprising 0.5% BSA, wherein the second dried blood sample is collected from the patient with a microsampling device following administration of the HCV therapeutic regimen to the patient; (d) detecting at least one anti-HCV antibody to at least one HCV encoded antigen in the eluted second dried blood sample, wherein the HCV therapeutic regimen is identified as having a therapeutic effect on HCV infection if the anti-HCV antibody levels in the second dried blood sample following the administration of the HCV therapeutic regimen are reduced compared to the anti-HCV antibody levels observed in step (b). In some embodiments, the buffer solution further comprises Phosphate Buffer Saline.

In any of the above embodiments, the HCV therapeutic regimen comprises one or more HCV inhibitors disclosed herein and/or other HCV inhibitors known in the art.

### Kits

Also disclosed herein are kits for detecting the presence of Hepatitis C virus (HCV) in a dried biological fluid sample. In some embodiments, the kits comprise a microsampling device, a lysis buffer, reverse transcriptase, and optionally a primer pair that specifically hybridizes to the 5′ UTR of the HCV genome. In certain embodiments, the kits comprise a detectably labelled probe that specifically hybridizes to the 5′ UTR of the HCV genome.

Additionally or alternatively, in some embodiments, the kits comprise a microsampling device, PBS solution comprising 0.5% BSA, and a detectably labelled secondary antibody that specifically binds to an anti-HCV primary antibody. In certain embodiments, the kits further comprise a solid substrate comprising wells coated with at least one HCV encoded antigen selected from the group consisting of c22-3, c200, and NS5.

In any of the above embodiments of the kits of the present technology, the microsampling device is a MITRA® tip.

In some embodiments, the kits comprise a primer pair that is capable of specifically hybridizing to the 5′ UTR of the HCV genome. Additionally or alternatively, in some embodiments, the kits provide a detectably labelled nucleic acid probe that specifically hybridizes to a sequence located within the region that is amplified by the primer pair.

In some embodiments, the kits may comprise a plurality of microsampling devices, each having a hollow holder at the proximal end and an absorbent tip at the distal end. The absorbent tip comprises a hydrophilic, polymeric material configured to absorb 30 microliters or less of blood within about 10 seconds or less. The kit also includes a container having a plurality of compartments. Each compartment is configured to releasably engage a microsampling device. The container is configured to prevent the absorbent tips of the microsampling devices from abutting the compartment within which the microsampling device is placed.

Additionally or alternatively, in certain embodiments, the kits may include a plurality of access ports with each port associated with an individual compartment. Each port is located to allow printing onto the holder of a microsampling device present within the compartment with which the port is associated. In certain embodiments, the holder of a microsampling device has a plurality of ribs extending along a length of the holder with the ribs configured to keep the absorbent tip from contacting walls of the container. The container preferably has two parts configured to form tubular shaped compartments. The container may have a first part with a plurality of elongated mounting protrusions each extending along a portion of a different compartment. The hollow end of the holder of the microsampling, device fits onto the mounting protrusion to releasably fasten the holder onto the mounting protrusion.

In some embodiments, the kit comprises liquid medium containing the at least one target-specific nucleic acid probe in a concentration of 250 nM or less. With such a kit, the probes are provided in the required amount to perform reliable multiplex detection reactions according to the present technology.

In some embodiments, the kits further comprise buffers, enzymes having polymerase activity, enzymes having polymerase activity and lacking 5′→3′ exonuclease activity or both 5′→3′ and 3′→5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of target nucleic acid sequences corresponding to HCV.

In one embodiment, the kits of the present technology further comprise a positive control nucleic acid sequence (e.g., HCV Quantitation Standard (QS) RNA molecules, such as those used in COBAS® AmpliPrep/COBAS® TaqMan® HCV Test) and a negative control nucleic acid sequence to ensure the integrity of the assay during experimental runs. Additionally or alternatively, in some embodiments, the kits of the present technology further comprise an anti-HCV positive-control biological sample and an anti-HCV negative-control biological sample. The kit may also comprise instructions for use, software for automated analysis, containers, packages such as packaging intended for commercial sale and the like.

The kit may further comprise one or more of: wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

### Examples

### Example 1: Detection of HCV with MITRA® Tips

Methods for Detecting HCV RNA.

A total of 79 human subjects were enrolled in the study. Conventional plasma or serum draws having a volume of at least 1 mL were obtained from each subject. Further, 20 μL of blood was collected from each subject with a MITRA® Tip collection device via fingerstick. The absorbent tips of the MITRA® Tip collection devices were then placed in 0.7 mL RLT buffer (Qiagen Inc., Valencia, Calif.) for at least 30 minutes at 37° C. with shaking at 800 rpm to elute the dried blood samples. Buffer RLT is a lysis buffer comprising a high concentration of guanidine isothiocyanate, and was used to lyse cells and tissues prior to RNA isolation. The absorbent tips were subsequently removed from the RLT buffer and 0.65 mL of each eluted dried blood sample were transferred to sample input tubes for testing on the COBAS® AmpliPrep/COBAS® TaqMan® Instrument. Specimen preparation, reverse transcription, and PCR amplification for each sample (MITRA® Tip and conventional blood drawn samples) were carried out on the COBAS® AmpliPrep/COBAS® TaqMan® platform in accordance with the manufacturer's instructions.

Briefly, HCV virus particles, if present in the sample, were lysed by incubation at elevated temperature with a protease and chaotropic lysis/binding buffer to release nucleic acids and protect the released HCV RNA from RNases. Protease and a known number of HCV Quantitation Standard (QS) RNA molecules were introduced into each specimen along with the lysis reagent and magnetic glass particles. Subsequently, the mixture was incubated and the HCV RNA (if present) and HCV QS RNA bound to the surface of the magnetic glass particles. Unbound substances, such as salts, proteins and other cellular impurities, were removed by washing the magnetic glass particles. After separating the magnetic glass particles and completing the washing steps, the adsorbed nucleic acids were eluted at an elevated temperature with an aqueous solution. Each processed specimen, containing the magnetic glass particles as well as released HCV RNA and HCV QS RNA, was added to the amplification mixture and transferred to the COBAS® TaqMan® Analyzer.

The COBAS® AmpliPrep/COBAS® TaqMan® HCV assay uses reverse transcription of HCV RNA to complementary DNA (cDNA) and PCR amplification of cDNA using primers that define a sequence within the highly conserved region of the 5′-untranslated region of the HCV genome and detectably labelled probes that also hybridize to the sequence defined by the primer pair. The nucleotide sequence of the primers has been optimized to yield comparable amplification of HCV genotypes 1 through 6. The reverse transcription and PCR amplification reaction was performed with an optimized blend of thermostable Z05 and Z05D DNA polymerases, which have both reverse transcriptase and DNA polymerase activity in the presence of manganese (Mn²⁺) and under the appropriate buffer conditions.

Processed specimens were added to the amplification mixture in amplification tubes (K-tubes) where both reverse transcription and PCR amplification occurred. The reaction mixture was heated to allow a downstream primer to anneal specifically to the HCV target RNA and to the HCV QS RNA. Following reverse transcription of the HCV target RNA and the HCV QS RNA, the reaction mixture was heated to denature the RNA:cDNA hybrid and to expose the specific primer target sequences. Upon cooling of the reaction mixture, the primers annealed to the target HCV DNA and the thermostable DNA Polymerases (Z05 and Z05D) in the presence of Mn²⁺ and excess deoxynucleotide triphosphates (dNTPs), extended the annealed primers along the target template to produce double-stranded DNA amplicons. The required number of cycles was preprogrammed into the COBAS® TaqMan® Analyzer or COBAS® TaqMan® 48 Analyzer.

Methods for Detecting Anti-HCV Antibodies.

A total of 242 human subjects were enrolled in the study. Conventional blood draws having a volume of at least 1 mL were obtained from each subject. Further, 20 μL of blood was collected from each subject with a MITRA® Tip collection device via fingerstick. The absorbent tips of the MITRA® Tip collection devices were then placed in 0.35 mL PBS/0.5% BSA overnight at 2-8° C. to elute the dried blood samples. The absorbent tips were subsequently removed from the buffer and each eluted dried blood sample was placed on the VITROS ECi/ECiQ Immunodiagnostics System (Ortho-Clinical Diagnostics, U.K.) for testing. Ten simulated fingerstick samples were used as negative controls. Immunoassays for each sample (MITRA® Tip and conventional blood drawn samples) were carried out on the VITROS ECi/ECiQ Immunodiagnostics System in accordance with the manufacturer's instructions.

Briefly, samples were transferred into wells coated with HCV recombinant antigens c22-3, c200, and NS5, allowing an HCV antibody (if present) in the sample to specifically bind to one or more of the HCV recombinant antigens. Unbound sample was removed by wash steps. The samples were then incubated with horseradish peroxidase (HRP)-labeled antibody conjugate (mouse monoclonal anti-human IgG), which binds to any human IgG captured on the well. Unbound conjugate was removed by wash steps. A reagent containing luminogenic substrates (a luminol derivative and a peracid salt) and an electron transfer agent, was added to the wells and the bound HRP conjugate was measured by a luminescent reaction. The amount of bound HRP conjugate was indicative of the level of anti-HCV present in the sample. Results were calculated as the ratio of a normalized signal relative to the cutoff value (signal/cutoff, s/c). A ratio<0.90 is considered a negative result, a ratio≥1.00 is a positive result, and a ratio between 0.9 to 0.99 is considered equivocal.

Results.

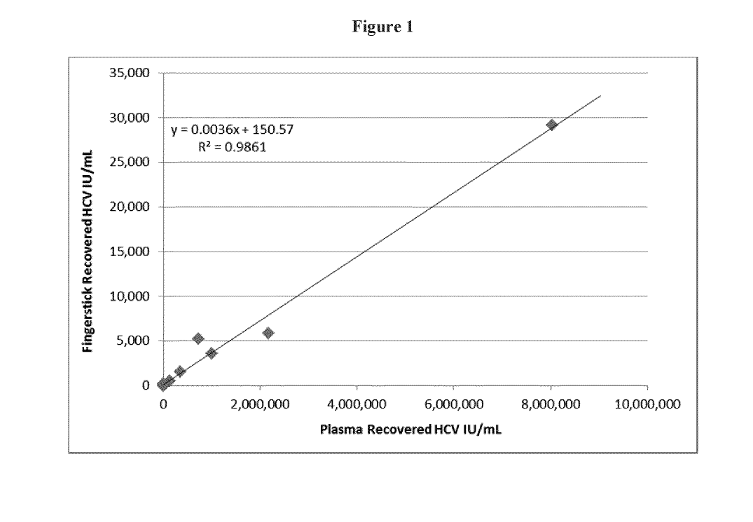

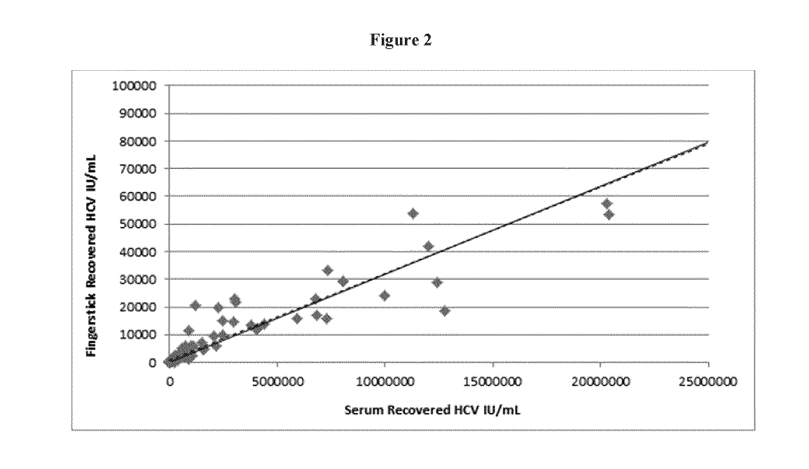

Both Table 1 and FIG. 1 provide a sampling of data from 32 of the 79 subjects, which demonstrates a complete concordance between HCV RNA detection in dried blood samples collected on a MITRA® tip collection device via fingerstick patients and HCV RNA detection in the whole blood samples from the same patients collected using conventional blood draws (R²=0.98). Table 1 also demonstrates that the HCV detection methods of the present technology are capable of reliably detecting viral loads at least as low as 126 IU/mL in small-volume dried blood samples obtained with a MITRA® Tip collection device via fingerstick. These results are remarkable given that assay sensitivity and precision are expected to decline with the reduction in sample volume. See Chen et al., Clin. Chem. 53:1962-1965 (2007). Previous studies have reported that HCV RNA quantitation in 10-μL finger-prick capillary whole blood samples is appropriate for monitoring viral loads of >900 IU/mL. Bruns et al., J. Clin. Microbiol. 47(10): 3231-3240 (2009). FIG. 2 shows a correlation of recovered viral loads between fingerstick recovered HCV and serum recovered HCV.

Table 2 provide a sampling of data from 45 of the 242 subjects, which demonstrates the concordance between anti-HCV antibody detection in dried blood samples collected on a MITRA® tip collection device via fingerstick and anti-HCV antibody detection in the whole blood samples from the same patients collected using conventional blood draws.

Table 3 shows that samples collected on a MITRA® tip collection device are also unexpectedly stable over time at room temperature (18-24° C.), regardless of whether the fluid collected was serum/plasma or whole blood. This is a valuable technical benefit because biological samples are usually not stable when stored at room temperature for extended periods of time. The present result show that even after two months of storage at room temperature, the samples are still able to provide accurate, reproducible results.

TABLE 1
HCV RNA Detection

| | | | | Finger Stick | |
|---|---|---|---|---|---|
| | Plasma | | | (Whole Blood) | |
| Plasma | Sample Concentration | | Fingerstick | Sample Concentration | |
| Sample ID | IU/mL | Log IU/mL | Sample ID | IU/mL | Log IU/mL |
| 1 | ND | NEG | 1 | ND | NEG |
| 2 | ND | NEG | 2 | ND | NEG |
| 3 | ND | NEG | 3 | ND | NEG |
| 4 | ND | NEG | 4 | ND | NEG |
| 5 | <15 | <1.18 | 5 | ND | NEG |
| 6 | <15 | <1.18 | 6 | ND | NEG |
| 7 | 15.9 | 1.20 | 7 | ND | NEG |
| 8 | 20 | 1.30 | 8 | ND | NEG |
| 9 | 30 | 1.47 | 9 | ND | NEG |
| 10 | 119 | 2.08 | 10 | <15 | <1.18 |
| 11 | 858 | 2.93 | 11 | <15 | <1.18 |
| 12 | 1,160 | 3.06 | 12 | ND | NEG |
| 13 | 2,228 | 3.35 | 13 | <15 | <1.18 |
| 14 | 6,740 | 3.83 | 14 | 185 | 2.27 |
| 15 | 8,980 | 3.95 | 15 | 126 | 2.10 |
| 16 | 16,500 | 4.22 | 16 | 137 | 2.14 |
| 17 | 48,900 | 4.69 | 17 | 238 | 2.38 |
| 18 | 69,100 | 4.84 | 18 | 246 | 2.39 |
| 19 | 97,000 | 4.99 | 19 | 1,420 | 3.15 |
| 20 | 137,000 | 5.14 | 20 | 741 | 2.87 |
| 21 | 141,000 | 5.15 | 21 | 545 | 2.74 |
| 22 | 144,000 | 5.16 | 22 | 472 | 2.67 |
| 23 | 222,000 | 5.35 | 23 | 2,510 | 3.40 |
| 24 | 300,000 | 5.48 | 24 | 2,400 | 3.38 |
| 25 | 317,000 | 5.50 | 25 | 1,130 | 3.05 |
| 26 | 327,000 | 5.51 | 26 | 2,010 | 3.30 |
| 27 | 359,000 | 5.56 | 27 | 1,560 | 3.19 |
| 28 | 369,000 | 5.57 | 28 | 2,970 | 3.47 |
| 29 | 397,000 | 5.60 | 29 | 873 | 2.94 |
| 30 | 731,000 | 5.86 | 30 | 5,220 | 3.72 |
| 31 | 1,000,000 | 6.00 | 31 | 3,610 | 3.56 |
| 32 | 2,170,000 | 6.34 | 32 | 5,860 | 3.77 |

TABLE 2
Anti-HCV Antibody Detection

| | Plasma | | Finger- | Fingerstick (WB) | |
|---|---|---|---|---|---|
| Plasma | HCV Antibody | | stick | HCV Antibody | |
| Sample ID | Result | Interpretation | Sample ID | Result | Interpretation |
| 1 | 27.6 | Reactive | 1 | 17.3 | Reactive |
| 2 | 0.06 | Negative | 2 | 1.41 | Reactive |
| | 0.05 | Negative | | 0.69 | Negative |
| 3 | 24.5 | Reactive | 3 | 7.51 | Reactive |
| 4 | 23.5 | Reactive | 4 | 13.4 | Reactive |
| 5 | 23.6 | Reactive | 5 | 3.58 | Reactive |
| 6 | 27.4 | Reactive | 6 | 18.9 | Reactive |
| 7 | 23.0 | Reactive | 7 | 2.77 | Reactive |
| 8 | 24.5 | Reactive | 8 | 14.8 | Reactive |
| 9 | 19.3 | Reactive | 9 | 2.12 | Reactive |
| | 19.9 | Reactive | | 2.03 | Reactive |
| 10 | 18.3 | Reactive | 10 | 15.6 | Reactive |
| 11 | 19.3 | Reactive | 11 | 12.0 | Reactive |
| 12 | 21.9 | Reactive | 12 | 8.0 | Reactive |
| 13 | 0.03 | Non reactive | 13 | 0.33 | Negative |
| 14 | 0.06 | Non reactive | 14 | 1.11 | Reactive |
| 15 | 0.37 | Non reactive | 15 | 0.87 | Negative |
| | | | | 0.86 | Negative |
| 16 | 3.27 | Reactive | 16 | 0.44 | Negative |
| 17 | 13.0 | Reactive | 17 | 1.27 | Reactive |
| 18 | 15.1 | Reactive | 18 | 1.10 | Reactive |
| 19 | 17.2 | Reactive | 19 | 7.63 | Reactive |
| 20 | 18.3 | Reactive | 20 | 14.4 | Reactive |
| 21 | 18.7 | Reactive | 21 | 1.10 | Reactive |
| 22 | 18.8 | Reactive | 22 | 0.67 | Negative |
| | | | | 0.71 | Negative |
| 23 | 19.1 | Reactive | 23 | 9.06 | Reactive |
| 24 | 19.3 | Reactive | 24 | 1.52 | Reactive |
| 25 | 19.3 | Reactive | 25 | 10.6 | Reactive |
| 26 | 20.1 | Reactive | 26 | 23.8 | Reactive |
| 27 | 20.2 | Reactive | 27 | 1.10 | Reactive |
| 28 | 20.3 | Reactive | 28 | 6.81 | Reactive |
| 29 | 20.4 | Reactive | 29 | 0.81 | Negative |
| | | | | 1.41 | Reactive |
| 30 | 21.2 | Reactive | 30 | 5.46 | Reactive |
| 31 | 21.3 | Reactive | 31 | 1.88 | Reactive |
| 32 | 21.4 | Reactive | 32 | 2.10 | Reactive |
| 33 | 21.7 | Reactive | 33 | 1.73 | Reactive |
| 34 | 21.7 | Reactive | 34 | 27.3 | Reactive |
| 35 | 21.8 | Reactive | 35 | 1.37 | Reactive |
| 36 | 21.9 | Reactive | 36 | 5.0 | Reactive |
| 37 | 22.4 | Reactive | 37 | 1.15 | Reactive |
| 38 | 22.6 | Reactive | 38 | 10.2 | Reactive |
| 39 | 23.0 | Reactive | 39 | 2.09 | Reactive |
| 40 | 23.5 | Reactive | 40 | 10.5 | Reactive |
| 41 | 23.6 | Reactive | 41 | 2.02 | Reactive |
| 42 | 24.5 | Reactive | 42 | 6.59 | Reactive |
| 43 | 24.5 | Reactive | 43 | 13.9 | Reactive |
| 44 | 27.4 | Reactive | 44 | 19.8 | Reactive |
| 45 | 27.6 | Reactive | 45 | 18.3 | Reactive |
| Negative | 0.08 | Negative | PBS/ | 0.00 | Negative |
| Plasma | 0.06 | Negative | 0.5% BSA | 0.00 | Negative |

TABLE 3
Stability of Anti-HCV Antibody and Detection Over Time

| | | Plasma/Serum | | | FINGERSTICK (WB) | |
|---|---|---|---|---|---|---|
| Fingerstick | Date | HCV Ab | | Date Mitra Collection | HCV Ab | |
| Sample ID | Drawn | Result | Interpretation | Device Tested | Result | Interpretation |
| 1 | Sep. 21, 2016 | 18.7 | Reactive | Oct. 13, 2016 | 1.10 | Reactive |
| | | | | Dec. 13, 2016 | 1.41 | Reactive |
| 2 | Sep. 27, 2016 | 30.3 | Reactive | Oct. 13, 2016 | 17.9 | Reactive |
| | | | | Dec. 13, 2016 | 13.1 | Reactive |
| 3 | Oct. 3, 2016 | 32.5 | Reactive | Oct. 13, 2016 | 24.9 | Reactive |
| | | | | Dec. 13, 2016 | 21.6 | Reactive |
| 4 | Oct. 5, 2016 | 30.3 | Reactive | Oct. 13, 2016 | 14.6 | Reactive |
| | | | | Dec. 13, 2016 | 10.2 | Reactive |
| 5 | Oct. 10, 2016 | 29 | Reactive | Oct. 13, 2016 | 16.3 | Reactive |
| | | | | Dec. 13, 2016 | 12.4 | Reactive |
| 6 | Oct. 10, 2016 | 27.1 | Reactive | Oct. 13, 2016 | 16.2 | Reactive |
| | | | | Dec. 13, 2016 | 14.3 | Reactive |
| 7 | Oct. 11, 2016 | 26.8 | Reactive | Nov. 3, 2016 | 11.0 | Reactive |
| | | | | Jan. 3, 2017 | 8.68 | Reactive |
| 8 | Oct. 13, 2016 | 18.8 | Reactive | Non. 3, 2016 | 0.67 | Non Reactive |
| | | | | Jan. 3, 2017 | 1.03 | Reactive |
| 9 | Oct. 18, 2016 | 13 | Reactive | Nov. 3, 2016 | 1.27 | Reactive |
| | | | | Jan. 3, 2017 | 1.09 | Reactive |
| 10 | Apr. 19, 2016 | 21.7 | Reactive | Nov. 3, 2016 | 1.73 | Reactive |
| | | | | Jan. 3, 2017 | 1.80 | Reactive |
| 11 | Oct. 25, 2016 | 20.3 | Reactive | Nov. 3, 2016 | 6.81 | Reactive |
| | | | | Jan. 3, 2017 | 5.20 | Reactive |
| 12 | Oct. 31, 2016 | 15.1 | Reactive | Nov. 3, 2016 | 1.10 | Reactive |
| | | | | Jan. 3, 2017 | 1.14 | Reactive |

These results demonstrate that the methods of the present technology are capable of detecting HCV RNA and/or anti-HCV antibodies in a small-volume dried biological fluid sample that is collected with a microsampling device (e.g., MITRA® Tip). The methods disclosed herein yielded results that were consistent with those observed with HCV detection methods employing conventional blood draws.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for detecting Hepatitis C virus (HCV) in a dried biological fluid sample comprising
(a) extracting ribonucleic acids from a dried biological fluid sample eluted from an absorbent tip of a microsampling device;
(b) reverse transcribing the extracted ribonucleic acids to generate a plurality of cDNA:RNA hybridization complexes;
(c) amplifying the cDNA:RNA hybridization complexes with a primer pair that specifically hybridizes to the 5′ UTR of the HCV genome to produce HCV amplicons; and
(d) detecting HCV in the dried biological fluid sample when the HCV amplicons produced in step (c) are detected.

2. The method of claim 1, wherein the HCV in the dried biological fluid sample has at least one genotype selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a.

3. The method of claim 1 or 2, wherein the dried biological fluid sample is dried plasma, dried serum, or dried whole blood.

4. The method of any one of claims 1-3, wherein the dried biological fluid sample on the absorbent tip of the microsampling device is collected from a patient via fingerstick.

5. The method of any one of claims 1-4, wherein elution of the dried biological fluid sample is performed by contacting the absorbent tip of the microsampling device with a lysis buffer.

6. The method of claim 5, wherein the lysis buffer comprises guanidine isothiocyanate, and optionally β-mercaptoethanol.

7. The method of any one of claims 1-6, wherein elution of the dried biological fluid sample is performed by contacting the absorbent tip of the microsampling device with the lysis buffer for at least 30 minutes at 37° C.

8. The method of any one of claims 1-7, wherein the microsampling device is a MITRA® tip.

9. The method of any one of claims 1-8, wherein the sample volume of the microsampling device is no more than 30 μL, no more than 25 μL, no more than 20 μL, no more than 15 μL, or no more than 10 μL.

10. The method of any one of claims 1-9, wherein the dried biological fluid sample is isolated from a patient exhibiting signs or symptoms of hepatitis, or a patient at risk for HCV infection.

11. The method of any one of claims 1-10, wherein the viral load of HCV in the dried biological fluid sample is at least 1-5 IU/mL, at least 5-10 IU/mL, at least 10-15 IU/mL, at least 15-20 IU/mL, at least 20-40 IU/mL, at least 40-60 IU/mL, at least 60-80 IU/mL, at least 80-100 IU/mL, at least 100-150 IU/mL, at least 150-200 IU/mL, at least 200-250 IU/mL, at least 250-300 IU/mL, at least 300-350 IU/mL, at least 350-400 IU/mL, at least 400-500 IU/mL, at least 500-600 IU/mL, at least 600-700 IU/mL, at least 700-800 IU/mL, at least 800-850 IU/mL, at least 850 IU/mL, or at least 900 IU/mL.

12. The method of any one of claims 1-11, further comprising contacting the cDNA:RNA hybridization complexes with a detectably labelled probe.

13. The method of claim 12, wherein the detectable label is a fluorescent reporter selected from the group consisting of 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, BODIPY® R-6G, BOPIPY® 530/550, BODIPY® FL, Brilliant Yellow, Cal Fluor Red 610® (CFR610), coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′, 5″-dibromopyrogallol-sulfonephthalein (Bromopyrogallol Red), 7-diethylamino-3-(4′-isothiocyanatophenyl)-4-methylcoumarin, di ethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, lanthamide phosphors, Malachite Green isothiocyanate, 4-methylumbelliferone, ortho cresolphthalein, nitrotyrosine, pararosaniline, Phenol Red, B-phycoerythrin, R-phycoerythrin, allophycocyanin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Quasar 670®, and VIC®.

14. The method of any one of claims 1-13, wherein the cDNA:RNA hybridization complexes are amplified with Z05 or Z05D DNA polymerases.

15. A method for detecting Hepatitis C virus (HCV) in a dried biological fluid sample comprising
(a) eluting a dried biological fluid sample from an absorbent tip of a microsampling device; and
(b) detecting HCV in the dried biological fluid sample when an anti-HCV antibody to at least one HCV encoded antigen is detected in the eluted dried biological fluid sample.

16. The method of claim 15, wherein the at least one HCV encoded antigen is selected from the group consisting of c22-3, c200, and NS5.

17. The method of claim 15 or 16, wherein the HCV in the dried biological fluid sample has at least one genotype selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a.

18. The method of any one of claims 15-17, wherein the dried biological fluid sample is dried plasma, dried serum, or dried whole blood.

19. The method of any one of claims 15-18, wherein the dried biological fluid sample on the absorbent tip of the microsampling device is collected from a patient via fingerstick.

20. The method of any one of claims 15-19, wherein elution of the dried biological fluid sample is performed by contacting the absorbent tip of the microsampling device with a mixture comprising Phosphate Buffer Saline and 0.5% BSA.

21. The method of claim 20, wherein elution of the dried biological fluid sample is performed by contacting the absorbent tip of the microsampling device with the mixture comprising Phosphate Buffer Saline and 0.5% BSA for at least 2 hours at room temperature, or overnight at 2-8° C.

22. The method of any one of claims 15-21, wherein the microsampling device is a MITRA® tip.

23. The method of any one of claims 15-22, wherein the sample volume of the microsampling device is no more than 30 no more than 25 no more than 20 μL, no more than 15 or no more than 10 μL.

24. The method of any one of claims 15-23, wherein the dried biological fluid sample is isolated from a patient exhibiting signs or symptoms of hepatitis, or a patient at risk for HCV infection.

25. A kit for detecting the presence of Hepatitis C virus (HCV) in a dried biological fluid sample comprising a microsampling device, a lysis buffer, reverse transcriptase, and optionally a primer pair that specifically hybridizes to the 5′ UTR of the HCV genome.

26. The kit of claim 25, further comprising a detectably labelled probe that specifically hybridizes to the 5′ UTR of the HCV genome.

27. A kit for detecting the presence of Hepatitis C virus (HCV) in a dried biological fluid sample comprising a microsampling device, PBS solution comprising 0.5% BSA, and a detectably labelled secondary antibody that specifically binds to an anti-HCV primary antibody.

28. The kit of claim 27, further comprising a solid substrate comprising wells coated with at least one HCV encoded antigen selected from the group consisting of c22-3, c200, and NS5.

29. The kit of any one of claims 25-28, wherein the microsampling device is a MITRA® tip.

30. A method for selecting a patient exhibiting hepatitis symptoms for treatment with a therapeutic agent that inhibits HCV infection comprising
(a) eluting a dried blood sample under conditions that result in the release of ribonucleic acids from blood cells, wherein the dried blood sample is collected from the patient with a microsampling device;
(b) isolating ribonucleic acids from the eluted dried blood sample;
(c) reverse transcribing the isolated ribonucleic acids to generate a plurality of cDNA:RNA hybridization complexes;
(d) amplifying the cDNA:RNA hybridization complexes with a primer pair that specifically hybridizes to the 5′ UTR of the HCV genome to produce fluorescently labelled HCV amplicons;
(e) detecting the fluorescently labelled HCV amplicons produced in step (d); and
(f) selecting the patient for treatment with a therapeutic agent that inhibits HCV infection, if the fluorescently labelled HCV amplicons are detected.

31. A method for selecting a patient exhibiting hepatitis symptoms for treatment with a therapeutic agent that inhibits HCV infection comprising
(a) eluting a dried blood sample with a buffer solution comprising 0.5% BSA, wherein the dried blood sample is collected from the patient with a microsampling device;
(b) detecting an anti-HCV antibody to at least one HCV encoded antigen in the eluted dried blood sample; and
(c) selecting the patient for treatment with a therapeutic agent that inhibits HCV infection, if the anti-HCV antibody is detected.

32. The method of claim 30 or 31, wherein the microsampling device is a MITRA® tip.

33. The method of any one of claims 30-32, wherein the therapeutic agent that inhibits HCV infection is one or more agents selected from the group consisting of interferon alfacon-1, pegylated and/or non-pegylated interferon alfa-2b, peginterferon alfa-2a, ribavirin, telaprevir, boceprevir, sofosbuvir, simeprevir, daclatasvir, velpatasvir, ombitasvir, paritaprevir, ritonavir, dasabuvir, ledipasvir, elbasvir, danoprevir, grazoprevir, GS-7977, β-interferon, γ-interferon, amantadine, and 3TC.

34. The method of any one of claims 30-33, wherein the HCV in the dried blood sample has at least one genotype selected from the group consisting of Genotype 1a, Genotype 1b, Genotype 2a, Genotype 2b, Genotype 2c, Genotype 2d, Genotype 3a, Genotype 3b, Genotype 3c, Genotype 3d, Genotype 3e, Genotype 3f, Genotype 4a, Genotype 4b, Genotype 4c, Genotype 4d, Genotype 4e, Genotype 4f, Genotype 4g, Genotype 4h, Genotype 4i, Genotype 4j, Genotype 5a, and Genotype 6a.

35. The method of any one of claims 30-34, wherein the at least one HCV encoded antigen is selected from the group consisting of c22-3, c200, and NS5.

36. A method for detecting Hepatitis C virus (HCV) in a dried biological fluid sample comprising isolating HCV RNA from a dried biological fluid sample eluted from an absorbent tip of a microsampling device with a lysis buffer.

37. The method of claim 36, wherein the HCV RNA is detected using reverse-transcription and real-time PCR.

38. The method of claim 36 or 37, wherein the lysis buffer comprises guanidine isothiocyanate, and optionally β-mercaptoethanol.

39. A method for detecting Hepatitis C virus (HCV) in a dried biological fluid sample comprising isolating an anti-HCV antibody from a dried biological fluid sample eluted from an absorbent tip of a microsampling device with a buffer solution comprising 0.5% BSA.

40. The method of claim 39, wherein the buffer solution comprises Phosphate Buffer Saline.

41. The method of claim 39 or 40, wherein the anti-HCV antibody is detected using ELISA.

42. The method of any one of claims 39-41, wherein the anti-HCV antibody binds to a HCV antigen selected from the group consisting of c22-3, c200, and NS5.
